(19) United States
(12) Patent Application Publication
(10) Pub. No.: US 2009/0088607 A1
(43) Pub. Date: Apr. 2, 2009

(54) CELL PHONE REMOTE DISEASE MANAGEMENT

(75) Inventor: John R. MURACA, Poland, NY (US)

Correspondence Address: STAAS & HALSEY LLP, SUITE 700, 1201 NEW YORK AVENUE, N.W., WASHINGTON, DC, 20005, US

(73) Assignee: VISUAL TELECOMMUNICATIONS NETWORK, INC., McLean, VA (US)

(21) Appl. No.: 12/239,906

(22) Filed: Sep. 29, 2008

Related U.S. Application Data
(60) Provisional application No. 60/976,199, filed on Sep. 28, 2007.

(51) Int. Cl. A61B 5/00; G06Q 50/00

(52) U.S. Cl. 600/300; 705/3

(57) ABSTRACT

A remote disease management system, method, and computer-readable medium includes a patient cell phone acquiring medical data and automatically transmitting the acquired data to a server. The patient cell phone acquires the medical data wirelessly or by manual input from remote sensors acquiring medical information about the patient, and automatically transmits the medical data to a server. The patient cell phone receives a text message from a physician workstation with medical questions and the patient cell phone receives responses to the medical questions from the patient and automatically transmits the responses to a server. Moreover, the patient cell phone transmits both the medical data received from the remote sensors and the responses to the medical questions to the server.

### CROSS-REFERENCE TO RELATED APPLICATION(S)

This application is related to U.S. provisional application entitled Cell Phone Remote Disease Management having Ser. No. 60/976,199, by John R. Muraca, filed Sep. 28, 2007 and incorporated by reference herein.

### BACKGROUND

1. Field

The embodiments discussed herein are directed to cell phone remote disease management.

2. Description of the Related Art

Advances in medicine and technology have vastly expanded the capacity of our health care system to prevent, diagnose, treat, and cure illness, In many cases, standard medical practice has not kept pace with these advances, resulting in a growing gap between what medical care could offer and what it does offer.

To meet the challenge of bringing the practice of medicine closer to advances in medical technology and practice, the realization of remote disease management includes the implementation of novel medical concepts, treatment and prevention paradigms, and technology tools in the process of patient care, to remotely manage traumatic brain injury (TBI), blood glucose, chronic obstructive pulmonary disease (COPD) and others. A component of remote disease management is the utilization of remote monitoring and remote care delivery tools. The realization of this vision includes the implementation of medical concepts and tools, including genetic devices, electronic medical records and remote patient monitoring and care delivery.

### SUMMARY

It is an aspect of the embodiments discussed herein to provide a novel, low cost, simple-functioning gateway using a communication device (for example, a cell phone) to enable transfer of data from multiple types of remote sensors via a service tier for delivery to a healthcare information system. The intent is to provide a low-cost means, using wireless technology, to interact with patients to acquire information concerning their well-being which is automatically sent to a central data base and, when necessary, alerts an attending physician of the need to directly intervene with the patient.

It is a further aspect of the embodiments to monitor remotely a patient's state of being. Monitoring remotely a patient's state of being is accomplished by monitoring a patient's response to direct, individualized, specific questions selected by the attending physician. In an aspect of the present invention, commercially available cell phone text technology (that is, text messaging) is used to communicate with the patient and to automate the sending of his/her response without the need to dial a number. Patient responses that are indicative of some deterioration in their condition are automatically alerted to the attending physician.

The above aspects can be attained by a system, method, and computer-readable medium that includes a patient cell phone acquiring medical data and automatically transmitting the acquired data to a server. In an aspect of the present invention, the patient cell phone acquires the medical data wirelessly or through manual entry from remote sensors acquiring medical information about the patient, and automatically transmits the medical data to a server. In another aspect of the present invention, the patient cell phone receives a text message from a physician workstation with medical questions and the patient cell phone receives responses to the medical questions from the patient and automatically transmits the responses to a server. In a further aspect of the present invention, the patient cell phone transmits both the medical data received from the remote sensors and the responses to the medical questions to the server.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

FIG. 1 is a diagram of a cell phone remote disease management system 100 according to the present invention.

FIG. 2 is a diagram 200 of an example of data flow according to the cell phone remote disease management system 100 of the present invention.

### DETAILED DESCRIPTION OF THE EMBODIMENTS

Embodiments of the present invention are disclosed with reference to VITEL NET™ (VITEL NET™, ViTel Net™, and Medvizer™ are trademarks of Visual Telecommunications, Inc.) but the present invention is not limited to such embodiments.

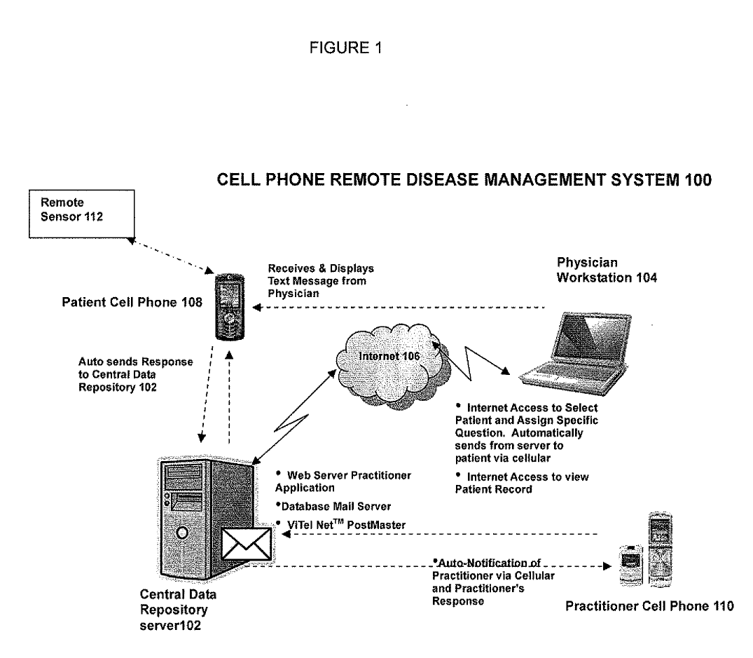

FIG. 1 is a diagram of a cell phone remote disease management system (CPRDM) 100 according to the present invention. By way of example, the cell phone remote disease management system 100 of FIG. 1 could be a traumatic brain injury (TBI) survey network.

As shown in the cell phone remote disease management system 100 of FIG. 1, a central data repository server 102 (which includes a central data repository) and a physician workstation 104 are in communication with each other by a network 106 such as the Internet. Also as shown in FIG. 1, a patient cell phone 108 is in communication with the physician workstation 104 and with the central data repository server 102, and a practitioner (physician) cell phone 110 is in communication with the central data repository server 102 102. In addition, a remote sensor 112 is in communication with the patient cell phone 108. Examples of a remote sensor 112 include a blood pressure monitor and a glucose monitor, but the present invention is not limited to these examples.

In an embodiment cell phone remote disease management system 100 of the present invention, communication between two or more of the central data repository server 102, the physician workstation 104, the network 106, the patient cell phone 108, and practitioner cell phone 1120, and the remote sensor 112 may be wireless communication, wired communication, or a combination thereof.

In an embodiment of the present invention, the communication between the remote sensor 112 and the patient cell phone 108 may be wireless communication such as Bluetooth. In another embodiment of the present invention, the communication may be through a patient entering data from the remote sensor 112 into the patient cell phone 108.

Referring again to the cell phone remote disease management system 100 of FIG. 1, embodiments of the present invention are discussed in further detail.

Physician Workstation 104:

A workstation, such as the ViTel Net™ Thin Client, may function as the physician workstation 104. The physician workstation 104 may be a mobile workstation or a workstation fixed in place. The physician workstation 104 includes network 106 access (such as Internet access) to select a patient and to assign a specific question to a patient, and to view a patient record. The physician workstation automatically forwards data or a question from the central data repository server 102 to the patient via the patient cell phone 108 through cellular communication.

The physician workstation 104 selects a patient, select an appropriate question from a list of pre-formatted questions, and automatically sends to the patient (for example, to the patient cell phone 102) as a text message using cellular technology.

Patient Cell Phone 108;

The patient cell phone 108 receives and displays incoming text messages from a physician, such as from physician workstation 104, and acquires and transmits data (such as medical data) to the central data repository server 102. An example of a patient cell phone 108 is a standard cell phone, which requires no modification or change to the communication capability of the standard cell phone.

The patient cell phone 108 may be used to acquire data (such as medical data) as either or both an acquisition device to acquire the data directly from a remote sensor 112 or as a data input device to receive the data as inputs from a patient manually entering the data into the patient cell phone 108. The data may include results (or readings) of medical tests performed by remote sensors 112 or responses to medically-related questions received from the physician workstation 104.

In an embodiment of the present invention, the patient cell phone 108 can be used as an acquisition device to acquire data from sensors such as home monitoring devices for blood pressure (blood pressure monitors), blood glucose (blood glucose monitors). If the patient cell phone 108 is enabled with wireless technology (for example, Bluetooth), then the patient cell phone 108 may interact directly with a home monitoring device to acquire data from the home monitoring device if the home monitoring device is also enabled with wireless technology. Otherwise, a patient may enter data from the home monitoring device manually into the patient cell phone 108 through an input such as a keyboard or touch screen.

In another embodiment of the present invention, the patient cell phone 108 can be used as a data input device to enable the patient to answer questions, for example related to protocols, received by the patient cell phone 108 and to forward the answers. The patient cell phone 108 receives and displays a text message from a physician, for example, from the physician workstation 104. The patient responds to a question by indicating 1, 2, 3, 4, or 5 Likert Scale using the patient cell phone 108. The patient's response is automatically sent to the central data repository server 102 without direct intervention by the patient. That is, the patient cell phone 108 auto-sends the response to the central data repository server 102.

In yet another embodiment of the present invention, the patient cell phone 108 can be used as both an acquisition device and as a data input device.

In one embodiment of the present invention, the remote sensors 112 may be located in a home such as the patient's home. The patient cell phone 108 (that is, the communication device) in the home (for example) transmits data such as readings received from the remote sensors 112 along with answers to health assessment questions gathered from the patient to a server such as the central data repository server 102 on the Internet broadband connectivity.

Central Data Repository Server 102;

The central data repository (or central call center) server 102 is executes a web server practitioner application, a data base mail server, and the ViTel Net™ PostMaster Database which receives incoming messages (for example, from the patient cell phone 108) and files to the appropriate patient record. A color identifier is assigned to each based upon the patient's response received from the patient cell phone 108. Reponses at the low end are categorized as green whereas those at the higher end receive a red identifier. An Automatic notification is sent to the attending physician via the practitioner cell phone 110 for patient responses categorized as yellow or red. A text message is sent to the practitioner's cell phone 110 indicating: patient name, patients medical record number (MRN), patient phone number, and color coded alert identifier.

The central data repository server 102 is, for example, Internet-based and the Internet-based data repository server 102 sends de-identified patient readings to, for example, a clinical system such as the Remote Monitoring Data Repository™ (RMDR™) clinical system behind a firewall via a web services interface.

The Remote Monitoring Data Repository™ (RMDR™) includes a database, which is structured to interact with other enterprise clinical systems (VitelNet™ Provides), and receives remotely monitored data points on patients and matches those readings to identified patient information.

Practitioner's Cell Phone 110:

The practitioner's cell phone 110 is also referred to as the physician's cell phone 110 and includes a standard cell phone without any modification of the cell phone communication capabilities. When the physician reads the text message, a notice is sent to the central data repository server 102 to indicate that the text message has been read. Moreover, the cell phone remote disease management system 100 of the present invention includes auto-notification of the practitioner by the central data repository server 102 via the practitioner's cell phone 110 and communication of the practitioner's response from the practitioner's cell phone 110 to the central data repository server 102.

Remote Devices 112:

The remote devices 112 include commercially-available devices, for example a blood glucose measurement device (glucometer), an oximeter, and weight scales, and others. In data transmission from the remote devices 112, the remote devices 112 transmit their readings wirelessly to a communications device, for example a cell phone such as the patient cell phone 108. These remote devices 112 are located, for example, in the patient's home. In another embodiment, the patient may manually enter the readings (i.e. medical data) taken by the remote devices 112 into the patient cell phone 102. In yet another embodiment, the remote devices 112 may wirelessly transmit the readings to the patient cell phone 108 and the patient may enter the readings manually into the patient cell phone 108.

The cell phone remote disease management system 100 of the present invention enhances patient-provider communication. The patient can see their daily readings reflected in the in-home data collection through a portal 102 (i.e., the Internet 106). A practitioner such as a Remote Monitoring nurse will view the patient readings in a portal designed to create a dashboard for managing many patients at once, such as the physician workstation 104.

Text Message Format
a. Develop text message format
b. Develop Likert Scale response format
c. Test sending messages to various cell phones
d. Access text message layout
e. Evaluate readability
f. Develop auto call-back feature upon selecting response MedVizer™ PostMaster and Database Server
a. Enable MedVizer™ PostMaster to receive cellular text messages
b. Create means for Patient Identification to be created via call in cell phone number linked to MRN in database
c. patient record

Auto Notification of Attending Physician
a. Develop means of categorizing patient data into alert areas
b. Develop means to automatically notify attending physician, via cell phone, when patient falls into “yellow” or “red” category.
   i. Name of Patient
   ii. Category of alert

Attending Physician Web Access Application to Send Text Message
a. Create Send Text Screens
   i. List of Patients
   ii. List of Questions
   iii. List Cell Phone Providers
   iv. List of Auto Notification

Attending Physician Web Access Application to view Record
a. Create Viewing Screens
   i. List of Patients
   ii. Alert Status
   iii. Individual Patient Record
   iv. Graphic Representation of results

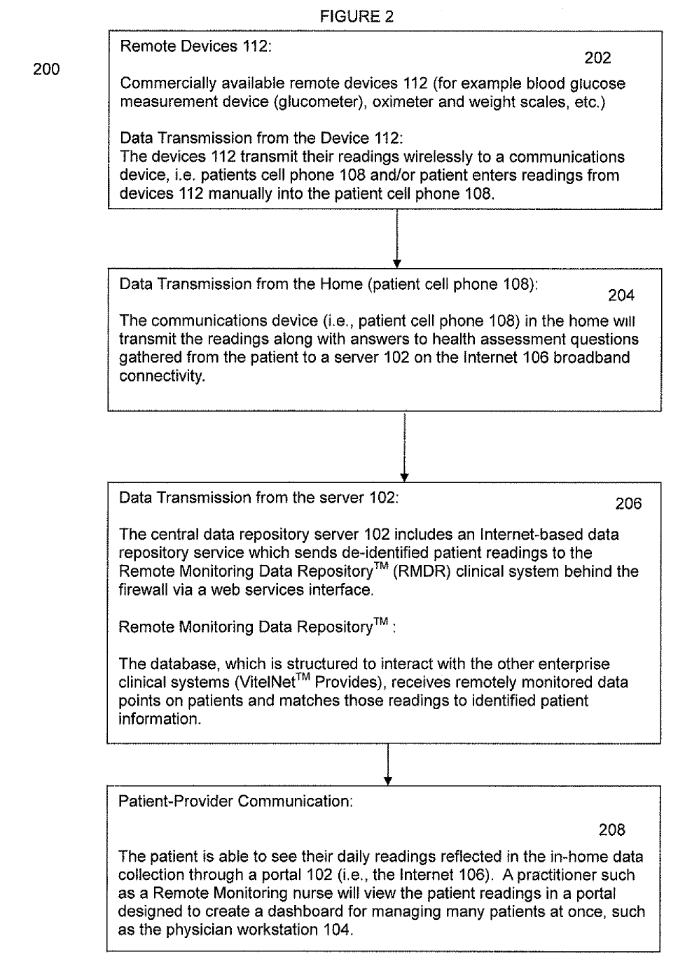

FIG. 2 is a diagram 200 of an example of data flow according to the cell phone remote disease management system 100 of the present invention.

As shown in 202 of FIG. 2, remote devices 112 may be commercially available remote devices 112 (for example blood glucose measurement device (glucometer), oximeter and weight scales, etc.) For data Transmission from the device 112, the devices 112 transmit their readings wirelessly to a communications device, i.e. patient cell phone 108. Alternatively, or in addition, the patient may enter the readings manually into the patient cell phone 108.

As shown in 204 of FIG. 2, in data transmission from the Home (patient cell phone 108), the communications device (i.e., patient cell phone 108) in the home will transmit the readings along with answers to health assessment questions gathered from the patient to a server 102 on the Internet 106 broadband connectivity.

As shown in 206 of FIG. 2, in data transmission from the server 102, the central data repository server 102 includes an Internet-based data repository service which sends de-identified patient readings to the Remote Monitoring Data Repository™ (RMDR) clinical system behind the firewall via a web services interface. The Remote Monitoring Data Repository™ includes a database, which is structured to interact with the other enterprise clinical systems (VitelNet™ Provides), which receives remotely monitored data points on patients and matches those readings to identified patient information.

As shown in 208 of FIG. 2, in patient-provider communication, the patient is able to see their daily readings reflected in the in-home data collection through a portal 102 (i.e., the Internet 106). A practitioner such as a Remote Monitoring nurse will view the patient readings in a portal designed to create a dashboard for managing many patients at once, such as the physician workstation 104.

In the above-mentioned embodiments of the present invention:
a. Cell phone text technology can be used to push a survey question to a patient.
b. Survey questions and response options can be developed for display and reading on a standard cell phone window.
c. Patient response to text message can be formatted using Likert scale; the higher number response indicates greater severity.
d. When the patient selects a response it can be automatically no intervention by the patient, sent to a central data repository using cellular technology.
e. The patient response can be automatically filed to the patient record and be assigned a color code (green, yellow, and red).
f. Yellow or red classification response can trigger the automatic sending of a text message to the attending physician indicating identity of the patient, phone, and priority of attention needed.
g. Non-response after a time period can trigger the automatic sending of a text message to the attending physician indicating identity of the patient, phone, and priority of attention needed.

The embodiments can be implemented in computing hardware (computing apparatus) and/or software, such as (in a non-limiting example) any computer that can store, retrieve, process and/or output data and/or communicate with other computers. The results produced can be displayed on a display of the computing hardware. A program/software implementing the embodiments may be recorded on computer-readable media comprising computer-readable recording media. The program/software implementing the embodiments may also be transmitted over transmission communication media. Examples of the computer-readable recording media include a magnetic recording apparatus, an optical disk, a magneto-optical disk, and/or a semiconductor memory (for example, RAM, ROM, etc.). Examples of the magnetic recording apparatus include a hard disk device (HDD), a flexible disk (FD), and a magnetic tape (MT). Examples of the optical disk include a DVD (Digital Versatile Disc), a DVD-RAM, a CD-ROM (Compact Disc-Read Only Memory), and a CD-R (Recordable)/RW. An example of communication media includes a carrier-wave signal.

Further, according to an aspect of the embodiments, any combinations of the described features, functions and/or operations can be provided.

The many features and advantages of the embodiments are apparent from the detailed specification and, thus, it is intended by the appended claims to cover all such features and advantages of the embodiments that fall within the true spirit and scope thereof. Further, since numerous modifications and changes will readily occur to those skilled in the art, it is not desired to limit the inventive embodiments to the exact construction and operation illustrated and described, and accordingly all suitable modifications and equivalents may be resorted to, falling within the scope thereof.

What is claimed is:

1. A method of remotely monitoring a patient, comprising:
acquiring data about the medical condition of the patient by a cell phone; and
automatically transmitting the data by the cell phone to a server.

2. The method of claim 1, wherein the acquiring comprising:
acquiring the data directly from a remote device.

3. The method of claim 1, wherein the acquiring comprising:
acquiring the data directly from a remote device by wireless communication.

4. The method of claim 1, wherein the acquiring comprising:
acquiring the data from a remote device by the patient manually entering the data into the cell phone.

5. The method of claim 1, wherein the acquiring comprising:
acquiring the data from the patient as responses to questions.

6. The method of claim 1, further comprising:
receiving by the patient cell phone from a practitioner a text message including medical questions; and
responding by the patient to the medical questions using the patient cell phone and automatically transmitting the response to the questions by the patient cell phone to a server.

7. The method of claim 1, further comprising:
transmitting survey questions and response options to a patient cell phone in a text message;
acquiring by the patient cell phone the patient response to the text message;
formatting the patient response by the patient cell phone using a Likert scale having a range of numbers with a higher number indicating a greater severity;
automatically forwarding the patient response by the patient cell phone to a central data repository using cellular technology and without intervention by the patient;
automatically filing by the central data repository the patient response to a patient record and assigning a color code, including green, yellow, and red;
automatically sending a text message to a physician's cell phone based upon the assigned color code of yellow or red, the text message indicating the identity of the patient, a phone number of the patient, and a priority of attention needed by the patient; and
automatically sending a text message to the physician's cell phone if the patient response is not received by the patient cell phone within a predetermined time period.

8. A computer readable medium storing a program which when executed by a computer caused to the computer to execute the functions of remotely monitoring a patient, comprising:
acquiring data about the medical condition of the patient by a cell phone; and
automatically transmitting the data by the cell phone to a server.

9. The computer-readable medium of claim 8, wherein the acquiring comprising:
acquiring the data directly from a remote device.

10. The computer-readable medium of claim 8, wherein the acquiring comprising:
acquiring the data directly from a remote device by wireless communication.

11. The computer-readable medium of claim 8, wherein the acquiring comprising,
acquiring the data from a remote device by the patient manually entering the data into the call phone.

12. The computer-readable medium of claim 8, wherein the acquiring comprising:
acquiring the data from the patient as responses to questions.

13. The computer-readable medium of claim 8, further comprising:
receiving by the patient cell phone from a practitioner a text message including medical questions; and
responding by the patient to the medical questions using the patient cell phone and automatically transmitting the response to the questions by the patient cell phone to a server.

14. The computer-readable medium of claim 8, further comprising:
transmitting survey questions and response options to a patient cell phone in a text message;
acquiring by the patient cell phone the patient response to the text message;
formatting the patient response by the patient cell phone using a Likert scale having a range of numbers with a higher number indicating a greater severity;
automatically forwarding the patient response by the patient cell phone to a central data repository using cellular technology and without intervention by the patient;
automatically filing by the central data repository the patient response to a patient record and assigning a color code, including green, yellow, and red;
automatically sending a text message to a physician's cell phone based upon the assigned color code of yellow or red, the text message indicating the identity of the patient, a phone number of the patient, and a priority of attention needed by the patient; and
automatically sending a text message to the physician's cell phone if the patient response is not received by the patient cell phone within a predetermined time period.

15. A cell phone for remotely monitoring a patient, comprising:
means for acquiring data about the medical condition of the patient by a cell phone; and
means for automatically transmitting the data by the cell phone to a server.

16. The cell phone of claim 15, wherein the means for acquiring comprising:
means for acquiring the data directly from a remote device.

17. The cell phone of claim 15, wherein the means for acquiring comprising:
means for acquiring the data directly from a remote device by wireless communication.

18. The cell phone of claim 15, wherein the means for acquiring comprising:
Means for acquiring the data by receiving manual input of the data from the patient.

19. The cell phone of claim 15, wherein the means for acquiring comprising:
means for acquiring the data from the patient as responses to questions.

20. The cell phone of claim 15, further comprising:
means for receiving by the patient cell phone from a practitioner a text message including medical questions; and
means for responding by the patient to the medical questions using the patient cell phone and automatically transmitting the response to the questions by the patient cell phone to a server.

21. The cell phone of claim 15, further comprising:
means for receiving survey questions and response options transmitted to the patient cell phone in a text message;
means for acquiring by the patient cell phone the patient response to the text message;
means for formatting the patient response by the patient cell phone using a Likert scale having a range of numbers with a higher number indicating a greater severity;
means for automatically forwarding the patient response by the patient cell phone to a central data repository using cellular technology and without intervention by the patient; and
means for automatically sending a text message to the physician's cell phone if the patient response is not received by the patient cell phone within a predetermined time period.

22. A remote disease management system, comprising:
a patient cell phone acquiring data about the medical condition of the patient and automatically transmitting the data; and
a server receiving the data and storing the data in a patient's medical record.

23. The remote disease management system of claim 22, further comprising:
a remote sensor acquiring readings about a medical condition of a patient, wherein the patient cell phone acquires data from the remote sensor wirelessly.

24. The remote disease management system of claim 22, wherein the patient cell phone receives input from a patient about the medical condition of the patient.

25. The remote disease management system of claim 22, wherein the patient cell phone receives readings about a medical condition of a patient by manual input into the patient cell phone.

26. The remote disease management system of claim 22, wherein the server categorizes the data and transmits the data.

27. The remote disease management system of claim 26, further comprising:
a physician's cell phone receiving the categorized data of the patient.

28. The remote disease management system of claim 22, further comprising:
a physician workstation transmitting a text message with medical questions to the patient cell phone, wherein the patient responding to the medical questions by the patient cell phone and transmitting the patient's responses to the server by the cell phone.

29. The remote disease management system of claim 28, wherein:
the physician's workstation transmitting survey questions and response options to a patient cell phone in a text message;
the patient cell phone acquiring the patient response to the text message;
the patient cell phone formatting the patient response using a Likert scale having a range of numbers with a higher number indicating a greater severity;
the patient cell phone automatically forwarding the patient response to a central data repository using cellular technology and without intervention by the patient;
the server comprising a central data repository automatically filing the patient response to a patient record and assigning a color code, including green, yellow, and red;
the server automatically sending a text message to the physician's cell phone based upon the assigned color code of yellow or red, the text message indicating the identity of the patient, a phone number of the patient, and a priority of attention needed by the patient; and
the server automatically sending a text message to the physician's cell phone if the patient response is not received by the patient cell phone within a predetermined time period.
